SEQUENCE LISTING (14 sequences)

SEQ ID NO: 1 - DNA, 62 bp, Artificial Sequence
  RXR-ALPHA bases 61 to 122 on exon 4
gcaaggacctgacctacacctgccgcgacaacaaggactgcctgattgacaagcggcagcgg

SEQ ID NO: 2 - DNA, 10 bp, Artificial Sequence
  RXR-ALPHA bases 61 to 70 on exon 4
gcaaggacct

SEQ ID NO: 3 - DNA, 28 bp, Artificial Sequence
  RXR-ALPHA bases 114 to 141 on exon 4
cggcagcggaaccggtgccagtactgcc

SEQ ID NO: 4 - DNA, 23 bp, Artificial Sequence
  RXR-ALPHA bases 44 to 66 on exon 4
cttcaagcggacggtgcgcaagg

SEQ ID NO: 5 - DNA, 23 bp, Artificial Sequence
  RXR-ALPHA bases 79 to 101 on exon 4
cctgccgcgacaacaaggactgc

SEQ ID NO: 6 - DNA, 23 bp, Artificial Sequence
  RXR-ALPHA bases 106 to 128 on exon 4
ttgacaagcggcagcggaaccgg

SEQ ID NO: 7 - DNA, 25 bp, Artificial Sequence
  RXR-ALPHA-gRNA-F1
accgcttcaagcggacggtgcgcag

SEQ ID NO: 8 - DNA, 25 bp, Artificial Sequence
  RXR-ALPHA-gRNA-R1
aaaactgcgcaccgtccgcttgaag

SEQ ID NO: 9 - DNA, 25 bp, Artificial Sequence
  RXR-ALPHA-gRNA-F2
accggcagtccttgttgtcgcggcg

SEQ ID NO: 10 - DNA, 25 bp, Artificial Sequence
  RXR-ALPHA-gRNA-R2
aaaacgccgcgacaacaaggactgc

SEQ ID NO: 11 - DNA, 25 bp, Artificial Sequence
  RXR-ALPHA-gRNA-F3
accgttgacaagcggcagcggaacg

SEQ ID NO: 12 - DNA, 25 bp, Artificial Sequence
  RXR-ALPHA-gRNA-R3
aaaacgttccgctgccgcttgtcaa

SEQ ID NO: 13 - DNA, 19 bp, Artificial Sequence
  RXR-ALPHA-iden-F1
aggccattccagggttctc

SEQ ID NO: 14 - DNA, 20 bp, Artificial Sequence
  RXR-ALPHA-iden-R1
ctgttgtccatctcgggtgt

(19) United States
(12) Patent Application Publication
(10) Pub. No.: US 2021/0222203 A1
(43) Pub. Date: Jul. 22, 2021

(54) RXR-ALPHA GENE KNOCKOUT CELL LINE WITH STABLE AND LOW RXR-ALPHA PROTEIN EXPRESSION AND PRODUCTION METHOD THEREOF

(71) Applicant: SHENZHEN CENTER FOR DISEASE CONTROL AND PREVENTION, SHENZHEN (CN)

(72) Inventors: JIANQING ZHANG, Shenzhen (CN); YUNXIU LI, Shenzhen (CN); YOUSHENG JIANG, Shenzhen (CN); DONGTING WU, Shenzhen (CN); JINLING PENG, Shenzhen (CN)

(21) Appl. No.: 17/135,916

(22) Filed: Dec. 28, 2020

Related U.S. Application Data
(63) Continuation of application No. PCT/CN2018/093484, filed on Jun. 28, 2018.

(51) Int. Cl. C12N 15/90

(52) U.S. Cl. CPC C12N 15/907; C12N 2510/00; C12N 2800/80; C12N 2310/20

(57) ABSTRACT

The present invention relates to the technical field of biological preparation, and particularly to a RXRα gene knockout cell line with stable and low RXRα protein expression. The cell line is a RXRα gene knockout cell line, in which non-3 integer multiple of bases are knocked out. The RXRα protein expression in the cells is significantly decreased and the genotype is stable to be passaged, thus greatly improving the stability of the cell line with low RXRα protein expression. This is conducive to the stable passage and continuous culture of the cell line. The present invention further provides a production method of the cell line.

### CROSS REFERENCE OF RELATED APPLICATIONS

The present application is a continuation-application of International (PCT) Patent Application No. PCT/CN2018/093484 filed on Jun. 28, 2018, the entire contents of which are hereby incorporated by reference.

### REFERENCE TO AN ELECTRONIC SEQUENCE LISTING

The contents of the electronic sequence listing (Untitled ST25; Size:3,229 bytes; and Date of Creation: Apr. 6, 2021) is herein incorporated by reference in its entirely.

### TECHNICAL FIELD

The present invention relates to the technical field of biological production, and particularly to a RXRα gene knockout cell line with stable and low RXRα protein expression.

### BACKGROUND

The mRNA of Homo sapiens retinoid X receptor (RXR) gene mainly includes three spliceosomes, namely RXRα (NM_002957.5), RXRβ (NM_001291920.1), and RXRγ (NM_001291921.1).

The intracellular RXRα receptor is able to bind to a variety of drugs, and closely associated with many human diseases including but not limited to tumors, diabetes, neurodegenerative diseases, and steatohepatitis. The construction of a cell line with high or low RXRα protein expression facilitates the research on the pathogenesis of RXRα receptor-related diseases and the development of effective drugs targeting these diseases. The CRISPR/Cas technology is used to direct the endonuclease cas9 protein by a sequence-specific guide RNA to a target site for cleavage, forming DNA double-stranded break (DSB). Such a DNA damage can initiate the repair mechanism in the cells, mainly by two pathways. One is the low-fidelity non-homologous end joining (NHEJ) pathway, where NHEJ is a main DNA break repair mechanism in the cell and this repair mechanism is very prone to errors, leading to the base deletion or insertion after repair, resulting in frameshift mutations, and ultimately achieving the purpose of gene knockout. The second DNA break repair pathway is homology-directed repair (HR). This repair mechanism based on homologous recombination has high fidelity, but low probability of occurrence.

As mentioned above, the effective construction of a cell line with stable and low RXRα protein expression is essential for the study of the biological effects of RXRα protein in organisms and the development of drugs for related diseases. The present invention intends to solve this problem.

### SUMMARY

In view of the problems existing in the prior art, a first object of the present invention is to provide a RXRα gene knockout cell line with stable and low RXRα protein expression. The cell line is a cell line (RXR/KO-SK-N-SH cells) obtained by knocking out a designed gene sequence in immortalized human neuroblastoma cells (SK-N-SH cells), where the knockout site includes bases 1-180 on exon 4 of the RXRα gene.

Specifically, the cell line is RXR/KO-SK-N-SH-7 # (or RXR/KO-SK-N-SH-35 #) or RXR/KO-SK-N-SH-15 # respectively. RXR/KO-SK-N-SH-7 # (or RXR/KO-SK-N-SH-35 #) is a homozygous cell line in which the RXRα allele is knocked out. RXR/KO-SK-N-SH-15 # is a cell line in which the RXRα gene is dual knocked out. The sequences knocked out are respectively bases 61-122 (62 bp), and bases 61-122 (62 bp), bases 61-70 (10 bp), and bases 114-141 (28 bp) on exon 4 of the RXRα gene, respectively,

(SEQ ID NO: 1)
GCAAGGACCT GACCTACACC TGCCGCGACA ACAAGGACTG
CCTGATTGAC AAGCGGCAGC GG;
(SEQ ID NO: 2)
GCAAGGACCT;
and
(SEQ ID NO: 3)
CGGCAGCGGA ACCGGTGCCA GTACTGCC.

As identified by gene sequencing and Western Blot of the cell line, the cell line constructed in the present invention is a RXRα gene knockout cell line, in which the expression level of RXRα protein is significantly reduced. The expression level of RXRα protein in the cell line is 13% or 17% (P<0.01) of that in the wild-type SK-N-SH cell respectively. The cell line is a homozygous cell line in which the RXRα allele is knocked out or a cell line in which the RXRα gene is dual knocked out. The cell line is stable in genotype, can stably and lowly express the mRNA and protein of RXRα receptor in the cells, and does not require regular screening and identification of protein expression level, which simplifies the procedure of cell construction. Therefore, the reliability, stability and research efficiency of the test materials for studying related functions of RXRα protein are greatly improved, and the absence of wild-type cells reduces the difficulty in proliferation and culture of cells with low RXRα protein expression. The cell line obtained in the present invention is free of wild-type cells, and the present solution has obvious advantages compared with the existing “liposome transfection technology” currently known internationally.

Another object of the present invention is to provide a method for constructing the cell line. The method comprises specifically:

(1) according to the RXRα gene sequence information, designing a CRISPR knockout gRNA sequence (3 sequences), constructing a gRNA expression vector, co-transfecting the gRNA expression vector and a cas9 expression vector into PK15 porcine kidney cells, and detecting the cleaving activity of gRNA in vitro at the cell level; and

(2) co-transfecting the gRNA expression vector and cas9 expression vector into SK-N-SH neuroblastoma cells by nucleofection, screening with G418 to obtain a cell clone, identifying by PCR and gene sequencing whether the cell clone is a cell clone where the RXRα gene is knocked out by knocking out non-3 integer multiple of bases, and finally identifying the expression level of RXRα protein by Western blot, to obtain a cell line with a stably heritable genotype and low RXRα protein expression.

Specifically, the production method includes the following steps:

(1) determination of target site for RXRα gene knockout and design of gRNA;

(2) construction of gRNA expression vector;

(3) identification of cleaving activity of gRNA, comprising: transfecting a liposome into PK15 porcine kidney cells, and identifying the cleaving activity of gRNA by T7E1 digestion;

(4) screening of RXRα gene knockout cell line, comprising: transfecting the gRNA expression vector and cas9 expression vector into SK-N-SH neuroblastoma cells by nucleofection, screening with G418, cloning and culturing; and

(5) identification of RXRα gene knockout cell line.

A preferred scheme in the step (1) is to select exon 4 shared by RXRα-a/b/c as the target knockout site according to the RXRα gene sequence, design CRISPR knockout gRNA by online CRISPR DESIGN software and synthesize a complementary DNA Oligos primer.

Compared with the prior art, the present application has the following beneficial effects.

In the present invention, a cell line with stable and low RXRα protein expression is effectively constructed. The expression level of RXRα protein in the cell line is 13% or 17% (P<0.01) of that in the wild-type SK-N-SH cell respectively. The cell line is a homozygous cell line in which the RXRα allele is knocked out or a cell line in which the RXRα gene is dual knocked out. The cell line is stable in genotype, can stably and lowly express the RXRα protein, and does not require regular screening and identification of protein expression level. Therefore, the reliability, and research efficiency of the test materials for studying related functions of RXRα protein are greatly improved, and the difficulty in proliferation and culture of cells with low RXRα protein expression is reduced.

(2) The cell line obtained in the present invention is free of wild-type cells, and the present solution has obvious advantages compared with the existing “liposome transfection technology” currently known internationally.

(3) The production method simplifies the procedure of cell construction, is simple and readily available, has good reproducibility, and is easy for industrial application.

### BRIEF DESCRIPTION OF THE DRAWINGS

FIG. 1 is a schematic diagram showing the choice of a knockout site.

FIG. 2 is a schematic diagram showing the design of a gRNA sequence.

FIG. 3 is a schematic diagram showing the cell morphology of PK15 pig kidney cells 17 hrs after transfection with EGFP.

FIG. 4 shows the expression of enhanced green fluorescent protein (EGFP) in PK15 porcine kidney cells 17 hrs after transfection with EGFP.

FIG. 5 is a flow chart of identification of the cleaving activity of gRNA plasmid.

FIG. 6 is a schematic diagram showing the results of identification of the activity of three gRNAs.

FIG. 7 is a schematic diagram showing the results of mixed digestion of the first batch of a first numbered cell clones with T7 endonuclease.

FIG. 8 is a schematic diagram showing the results of mixed digestion of the first batch of a second numbered cell clones with T7 endonuclease.

FIG. 9 is a schematic diagram showing the results of mixed digestion of the first batch of a third numbered cell clones with T7 endonuclease.

FIG. 10 is a schematic diagram showing the results of direct digestion of the first batch of cell clones with T7 endonuclease.

FIG. 11 is a schematic diagram showing the results of digestion of the second batch of cell clones with T7 endonuclease.

FIG. 12 is a schematic diagram showing the identification of relative expression of RXRα protein in the RXRα gene knockout cell line of the present invention, in which 1-3 represent wild-type cells; 4-6 represent RXRα gene knockout cells 7 #; and 7-10 represent RXRα gene knockout cells 15 #.

FIG. 13 is a schematic diagram showing the determination of relative expression of RXRα protein in the RXRα gene knockout cell line of the present invention, in which SK-N-SHis the wild-type cells; RXR/KO-SK-N-SH-7 # is the RXRα gene knockout cells 7 #; and RXR/KO-SK-N-SH-15 # is the RXRα gene knockout cells 15 #.

### DESCRIPTION OF THE EMBODIMENTS

The present invention will be further described in detail below by way of examples with reference to accompanying drawings, but implementation of the present invention is not limited thereto.

### Example 1

Determination of Target Site for RXRα Gene Knockout and Design of gRNA

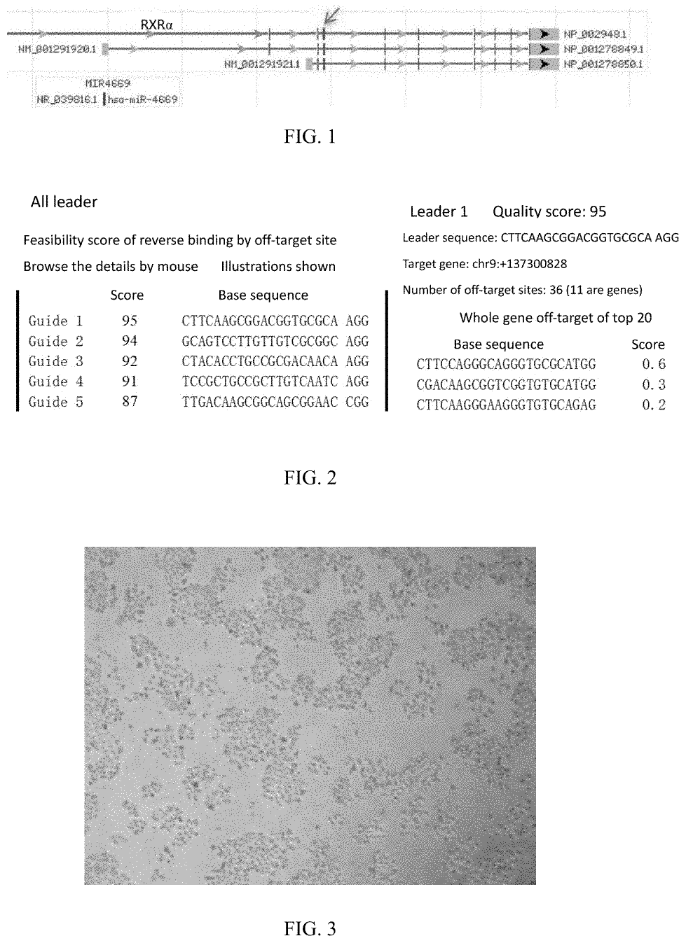

According to the RXRα gene sequence, exon 4 shared by RXRα-a/b/c was used as the target knockout sequence, and a CRISPR target site was designed. The knockout site selected was at the position indicated by the arrow in FIG. 1.

The selected target knockout site included bases 1-180 on exon 4 of the RXRα gene, and DNA Oligos primers were designed with bases 44-66 (CTTCAAGCGGACGGTGCGCAAGG) (SEQ ID NO: 4), bases 79-101 (CCTGCCGCGACAACAAGGACTGC) (SEQ ID NO: 5), and bases 106-128 (TTGACAAGCGGCAGCGGAACCGG) (SEQ ID NO: 6) respectively to synthesize double-stranded gRNAs.

CRISPR knockout gRNA was designed by online software and complementary DNA Oligos primers were synthesized. The design of gRNA sequence is shown in FIG. 2.

In FIG. 2, Guid #1-#5 are the target knockout sites with higher scores designed and selected by the software design. In this application, Guid #1, #2, #5 are used to design primers for gRNA synthesis, and their corresponding primer sequences are respectively RXRα-gRNA-F1/R1, RXRα-gRNA-F2/R2, RXRα-gRNA-F3/R3. The sequences are shown below:

| (SEQ ID NO: 7) | |
|---|---|
| RXRα-gRNA-F1: | ACCG CTTCAAGCGGACGGTGCGCA G |
| (SEQ ID NO: 8) | |
| RXRα-gRNA-R1: | AAAAC TGCGCACCGTCCGCTTGAAG |
| (SEQ ID NO: 9) | |
| RXRα-gRNA-F2: | ACCG GCAGTCCTTGTTGTCGCGGC G |
| (SEQ ID NO: 10) | |
| RXRα-gRNA-R2: | AAAAC GCCGCGACAACAAGGACTGC |
| (SEQ ID NO: 11) | |
| RXRα-gRNA-F3: | ACCG TTGACAAGCGGCAGCGGAACG |
| (SEQ ID NO: 12) | |
| RXRα-gRNA-R3: | AAAACGTTCCGCTGCCGCTTGTCAA |

When the gRNA activity is identified, the primers for amplification of RXRα gene are RXRα-iden-F1: AGGCCATTCCAGGGTTCTC (SEQ ID NO: 13), and RXRα-iden-R1: CTGTTGTCCATCTCGGGTGT (SEQ ID NO: 14). The total length of the RXRα sequence amplified with the primers is 748 bp. If gRNA is inactive, there is only one sequence with a total length of 748 bp after agarose gel electrophoresis. If gRNA has cleaving activity, it may cause gene deletion or insertion, and the PCR product can be digested by T7 endonuclease, so that multiple bands appear after agarose gel electrophoresis.

(2) Construction of gRNA Expression Vector

After gRNA synthesis, the primers were dissolved in water to give a concentration of 10 μM. Each 10 μL of the upstream and downstream primers was added, and incubated at 95° C. for 10 min, and then at room temperature for 30 min. then connect the backbone. A scaffold was then ligated, where the scaffold is a T vector with U6 promoter and gRNA tail, and the restriction site is BsaI. After the scaffold was prepared, it was ligated by T4 ligase at 16° C. for 4 hrs.

| | Amount used in a 20 μl |
|---|---|
| Reagent | reaction system |
| 10X T4 DNA Ligase Buffer* | 2 μl |
| Vector DNA (3 kb) | 50 ng (0.025 pmol) |
| Insert DNA (1 kb) | 50 ng (0.076 pmol) |
| Nuclease-free water | to 20 μl |
| T4 DNA Ligase | 1 μl |

After ligation, a single plasmid-containing clone was picked from the transformation plate, and sequenced to test the accuracy of the core sequence of the plasmid clone. A large number of plasmids were extracted from cells cultured on a shaker and frozen for storage.

Liposome Transfection

The cells were transiently transfected by CRISPR-Cas9 method, and the cleaving activity of the plasmid was verified by detecting the integrity of the target gene.

(3.1) PK15 porcine kidney cells were thawed in a 24-well plate (in 4 wells), and transiently transfected according to the instructions of invitrogen lipofectamin 2000. The cells in one well were transfected with EGFP containing plasmid and the transfection efficiency was observed; and the other three wells were co-transfected with the plasmid. The transfection parameters are shown below:

Liposome transfection parameters: Solution A

| | | | Opti-mem |
|---|---|---|---|
| | gRNA | cas9 | medium |
| gRNA1 | 300 ng | 200 ng | To 25 μl |
| gRNA2 | 300 ng | 200 ng | To 25 μl |
| gRNA3 | 300 ng | 200 ng | To 25 μl |
| EGFP | 500 ng | — | To 25 μl |

Solution B: lipo2000 (1.5 μl)+Opti-Mem (23.5 μl)=25 μl

Solution A and Solution B were mixed and transfected into the cells in the four wells.

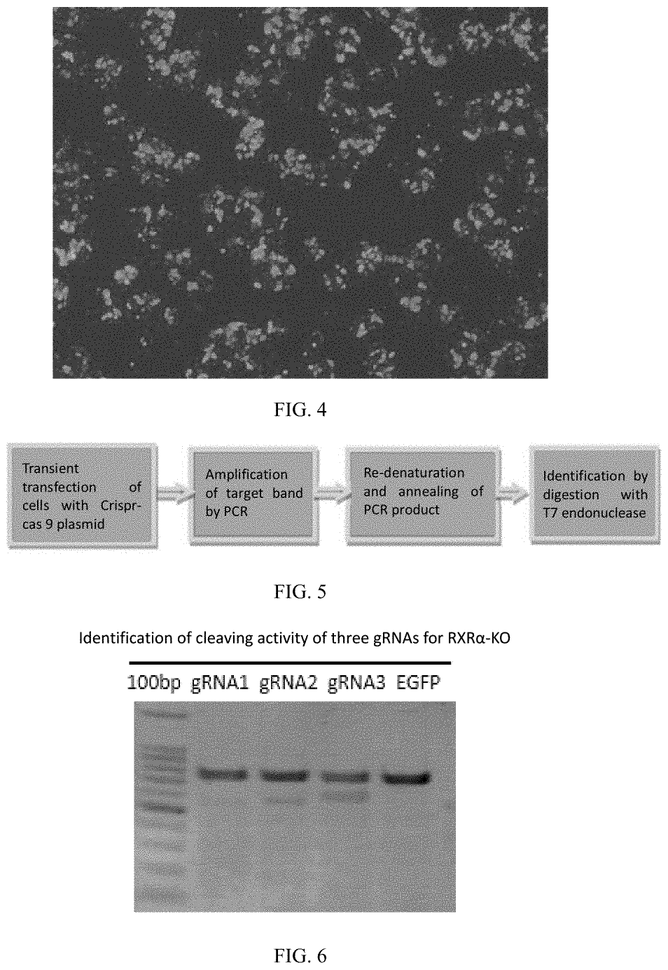

(3.2) The transfection with the target plasmid was determined based on the intensity of fluorescence expression from PK15 porcine kidney cells 17 hrs after transfection with EGFP containing plasmid. 17 hrs after transfection with EGFP containing plasmid, the PK15 porcine kidney cells were photographed under a fluorescence microscope. The expression of EGFP in the cells is shown in FIGS. 3 and 4. FIG. 3 shows the initial state of PK15 porcine kidney cells under a fluorescence microscope under natural light 17 hrs after transfection. It shows that the cell outline is clear after liposome transfection, indicating that liposome transfection has no obvious influence on the cell viability. FIG. 4 has the same field of microscope as that in FIG. 3, and shows the fluorescence state of EGFP at an excitation wavelength. By observing the ratio of fluorescence expression, the liposome transfection efficiency in the experimental group is reflected, and whether the cells in the experimental group can be used in subsequent activity identification is determined. The comparison of the two images shows that the transfection efficiency is over 80%, indicating that the PK15 porcine kidney cell can be used as a template for activity identification.

(3.3) Identification of Cleaving Activity of gRNA

The process was carried out following the flow chart as described in FIG. 5. 48 hrs after transfection, the cells were collected to extract DNA, and the activity was identified by digestion with T7 endonuclease. The principle for identifying the activity of gRNA containing vector by digestion with T7 endonuclease is as follows. In the CRISPR-Cas9 system, the sequence-specific guide RNA (gRNA) specifically recognizes the sequence at the knockout site of the RXRα gene, and direct the endonuclease cas9 protein to the target site for digestion, causing some base deletions or changes. This sequence is amplified, and then denatured and renatured. The wild-type sequence and the mutant sequence in the PCR product are annealed to form a double-stranded product. Due to the base change at the cleavage position, the bases are not complementary, forming a vesicle-like DNA structure. The T7 endonuclease can specifically cleave the vesicle-like single strand, and the PCR product is cleaved into two fragments. Therefore, the electrophoresis after digestion with T7 endonuclease shows multiple bands.

(3.3.1) Primer Information

TABLE 3-1
Information table of active primers

| Primer | | Fragment |
|---|---|---|
| name | Primer sequence | size |
| RXRα- | AGGCCATTCCAGGGTTCTC | 748 bp |
| iden-F1: | (SEQ ID NO: 13) | |
| RXRα- | CTG TTGTCCATCTCGGGTGT | |
| iden-R1: | (SEQ ID NO: 14) | |

(3.3.2) PCR Reaction System

Note: 1: Premix EXTaqase from TAKARA is used.

TABLE 3-2
PCR reaction system

| Reaction system | 1X | |
|---|---|---|
| EXTaqase | 10 | μl |
| Primer-F | 0.4 | μl |
| Primer-R | 0.4 | μl |
| DNA | 100 | ng |

| | |
|---|---|
| H₂O | To 20 μl |

(3.3.3) PCR Reaction Conditions

TABLE 3-3
PCR reaction procedure

| | | | |
|---|---|---|---|
| 98° C. | 5 | min | |
| 98° C. | 10 | sec | |
| 55° C. | 30 | sec | 35 cycles |
| 72° C. | 1 | min | |
| 72° C. | 5 | min | |
| 20° C. | 2 | min | |

The size of the bands after enzymatic digestion is shown in FIG. 6. The band sizes of the three gRNAs after enzymatic digestion are 536+212, 566+182, and 602+146, respectively.

This process was a PCR amplification procedure, including pre-denaturation at 98° C. for 5 min; 35 cycles of denaturation at 98° C. for 10 sec, annealing at 55° C. for 30 sec, and extension at 72° C. for 1 min; extension at 72° C. for 5 min to ensure the complete extension, and ensure the integrity of PCR amplification, following by standing at 20° C. for 2 min. The product was cooled and then stored to increase the stability of the PCR product.

(4) Screening of RXRα Gene Knockout Cell Lines

All of the three gRNAs were verified to have the gene cleaving activity and could be used to construct gene knockout cell lines. When the SK-N-SH cells were cultured and grown to reach 80%-90% confluence, the plasmid was nucleofected by a nucleofector according to a ratio of gRNA1:gRNA2:gRNA3:cas9:pcDNA3.1=1:1:1:2:1. The passage number of the cells was P3. The cell transfection was observed 24 hrs after transfection. When the confluence reached 80%-90%, G418 was added for screening on the following day, and a single clone was obtained 7 days later. A single clone was picked to culture in a 96-well plate, and expanded to culture in a 48-well plate when it was overgrown. When the confluence was 80%-90%, half of the cells were digested into a lysate for identification by PCR, and the remaining cells were cultured in the original wells.

(5) Identification of RXRα Gene Knockout Cell Line

(5.1) Identification of cell clones by digestion with T7 endonuclease

Whether the cells are gene knockout cell clones was determined by digestion with T7 endonuclease. PCR amplification and digestion with T7 endonuclease were carried out. A 20 μl system was used for PCR amplification. 2 μl of the PCR product was dotted, and the remaining 18 μl was divided into two groups. In one group, 9 μl was mixed with water at a ratio of 1:1, denatured, annealed, and then digested (directly digested) with T7 endonuclease. In the other group, 9 μl was mixed with wild-type PCR product at a ratio of 1:1 denatured, annealed, and then digested (mixed digested) with T7 endonuclease. If cleavable by direct digestion or by direct digestion and mixed digestion, the cells are heterozygous knockout cells. If failed to be cleaved by direct digestion but cleavable by mixed digestion, the cells are homozygous knockout cells. In view of the above, the cell line is indicated to be a RXRα gene knockout cell line. If failed to be cleaved by direct and mixed digestion, the cell line is not a RXRα gene knockout cell line.

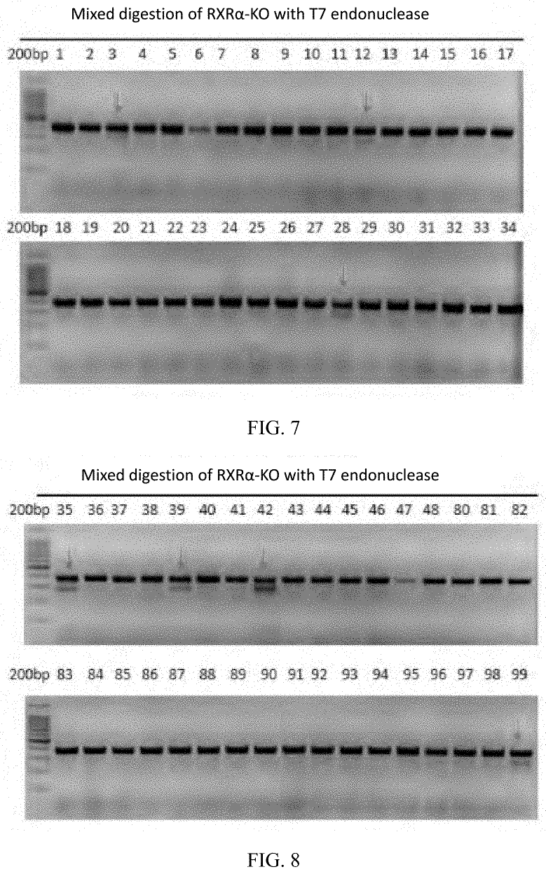

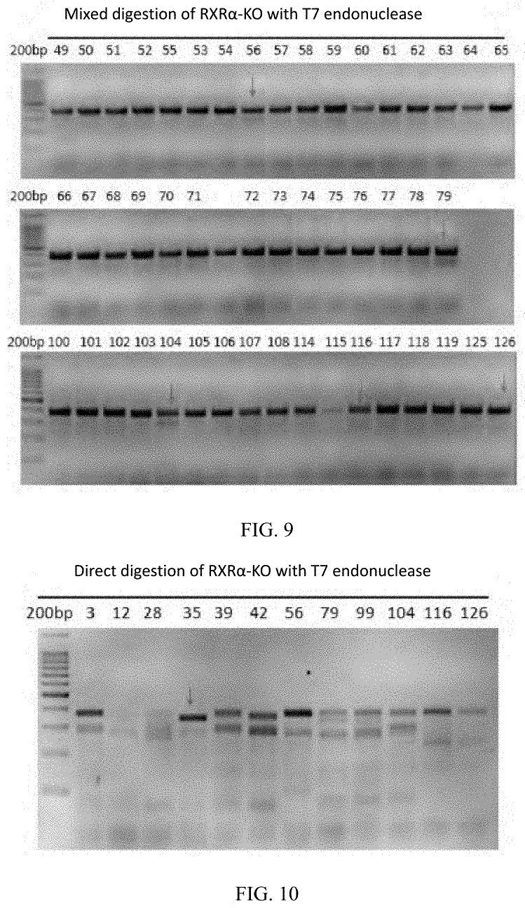

In this application, 250+ cell clones were picked up and 140 cell lines were identified. Because of the large number of identifications, mixed digestion was performed first. Clones cleavable by mixed digestion were then subjected to direct digestion. The results of mixed digestion of the first batch of cell clones with T7 endonuclease are shown in FIGS. 7 to 9, and the results of direct digestion of the first batch of cell clones with T7 endonuclease are shown in FIG. 10.

The results of identification by digestion with T7 endonuclease show that the cells of clone #35 are cleavable by mixed digestion with T7 endonuclease, but fail to be cleaved by direct digestion. Therefore, clone #35 is preliminarily identified as a homozygous knockout clone, and designated as RXR/KO-SK-N-SH-35 #.

One suspected positive clone RXR/KO-SK-N-SH-35 # is identified from one batch. Two suspected positive clones are identified from the other batch, which are respectively #7 and #15. The results of digestion with T7 endonuclease are shown in FIG. 11. The cell lines are designated as RXR/KO-SK-N-SH-7 # and RXR/KO-SK-N-SH-15 #.

(5.2) Gene Sequencing of Suspected Positive Clones

The PCR products of the cell lysates of suspected positive clones #7, #15, and #35 were connected to a T vector and sequenced. The genotypes of #7 and #35 are the same and clones #7 and #35 are both gene knockout clones in which bases 61-122 (62 bp) on exon 4 of the RXRα allele are deleted. Therefore, RXR/KO-SK-N-SH-7 # and RXR/KO-SK-N-SH-35 # are RXRα allele knockout homozygotes. #15 is a cell line in which the RXRα gene is dual knocked out, and the bases 61-122 (62 bp), 61-70 (10 bp) and 114-141 (28 bp) on exon 4 of the RXRα gene are deleted respectively. In all the three cell lines, bases of an integer multiple of a number other than 3 are knocked out, which can produce effective frameshift mutations, and finally achieve the effect of gene knockout.

(5.3) Identification of RXRα Protein Expression Level in RXRα Gene Knockout Positive Clones

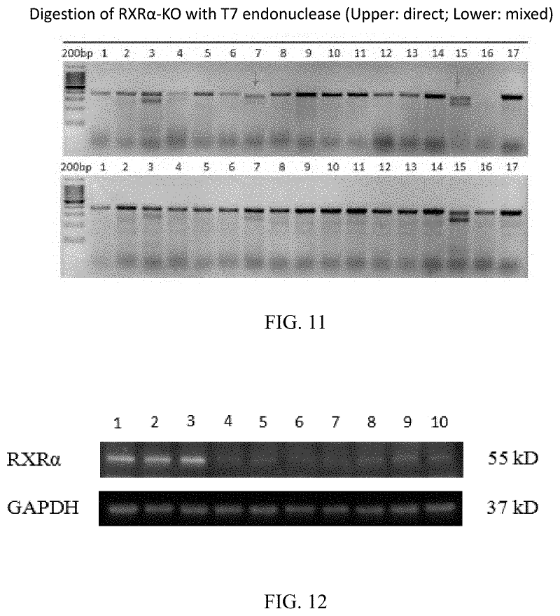

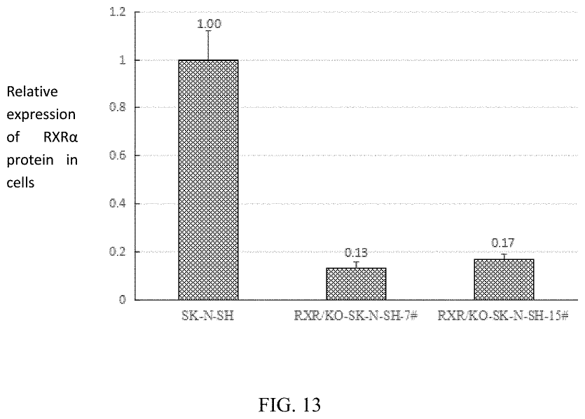

Gene sequencing showed that RXR/KO-SK-N-SH-7 # has the same genotype as RXR/KO-SK-N-SH-35 #, so in this section, only the cell lines RXR/KO-SK-N-SH-7 # and RXR/KO-SK-N-SH-15 # were identified by Western Blot. The identification results are shown in FIGS. 12 and 13. The expression of RXRα protein in #7 and #15 cells is significantly decreased, and the RXRα protein expression level in #7 and #15 cell lines is 13% and 17% of that in WT cells (P<0.01).

The above identification results show that through the research in this section, a RXRα gene knockout cell lines with stable and low expression of RXRα protein are successfully constructed. The cell line is RXR/KO-SK-N-SH-7 # (RXR/KO-SK-N-SH-35 #) or RXR/KO-SK-N-SH-15 #, which provides a test material for the study of low expression of RXRα protein in subsequent experiments.

The above examples are preferred embodiments of the present invention, but the embodiments of the present invention are not limited to thereto. Any other changes, modifications, replacements, combinations, and simplifications may be made without departing from the spirit and scope of the present invention, which are all embraced in the scope of the present invention.

1. A RXRα gene knockout cell line with stable and low RXRα protein expression, which is a gene knockout cell line obtained by knocking out a target site designed by CRISPR DESIGN software in immortalized human neuroblastoma cells, that is, SK-N-SH cells,
wherein the target site knocked out is bases 1-180 on exon 4 of the RXRα gene,
wherein the cell line is RXR/KO-SK-N-SH-7 # or RXR/KO-SK-N-SH-15 #; and
wherein RXR/KO-SK-N-SH-7 # is a homozygote in which the RXRα allele is knocked out; and
RXR/KO-SK-N-SH-15 # is a cell line in which the RXRα gene is dual knocked out, wherein bases 61-122 (62 bp), and bases 61-122 (62 bp), bases 61-70 (10 bp), and bases 114-141 (28 bp) on exon 4 of the RXRα gene are deleted respectively, and the sequences knocked out are respectively
(SEQ ID NO: 1)
GCAAGGACCT GACCTACACC TGCCGCGACA ACAAGGACTG
CCTGATTGAC AAGCGGCAGC GG;
(SEQ ID NO: 2)
GCAAGGACCT;
and
(SEQ ID NO: 3)
CGGCAGCGGA ACCGGTGCCA GTACTGCC.

2. A production method of a RXRα gene knockout cell line with stable and low RXRα protein expression according to claim 1, comprising
(1) according to the RXRα gene sequence information, designing CRISPR knockout gRNA, constructing a gRNA expression vector, co-transfecting the gRNA expression vector and a cas9 expression vector into PK15 porcine kidney cells, and detecting the cleaving activity of gRNA in vitro at the cell level; and
(2) co-transfecting the gRNA expression vector and cas9 expression vector into neuroblastoma cells SK-N-SH by nucleofection, screening with G418 to obtain a stable cell clone, identifying by PCR and gene sequencing whether the cell clone is a cell clone where the RXRα gene is knocked out by deleting bases of an integer multiple of a number other than 3, and finally identifying the expression level of RXRα protein by Western blot, to obtain a RXRα gene knockout cell line with a stably heritable genotype.

3. The production method according to claim 2, comprising the steps of:
(1) determination of target site for RXRα gene knockout and design of gRNA;
(2) construction of gRNA expression vector;
(3) identifying cleaving activity of gRNA, comprising: liposome transfection, and identification of the cleaving activity of gRNA by T7E1 digestion;
(4) screening of RXRα gene knockout cell line, comprising: transfecting the gRNA expression vector and cas9 expression vector into SK-N-SH neuroblastoma cells by nucleofection, screening with G418, cloning and culturing; and
(5) identification of RXRα gene knockout cell line.

4. The production method according to claim 3, wherein in Step (1), exon 4 shared by RXRα-a/b/c is selected as the site for knockout according to the RXRα gene sequence, and CRISPR knockout target site is designed, wherein the selected target site for knockout is bases 1-180 on exon 4 of the RXRα gene; and DNA Oligos primers are designed with bases 44-66

| | |
|---|---|
| CTTCAAGCGGACGGTGCGCAAGG, | (SEQ ID NO: 4) |

bases 79-101

| | |
|---|---|
| CCTGCCGCGACAACAAGGACTGC, | (SEQ ID NO: 5) |

and bases 106-128

| | |
|---|---|
| TTGACAAGCGGCAGCGGAACCGG | (SEQ ID NO: 6) |

respectively on exon 4 of the RXRα gene to synthesize double-stranded gRNAs; and
CRISPR knockout gRNA is designed by online software CRISPR DESIGN and complementary DNA Oligos primers are synthesized.

5. The production method according to claim 3, wherein in Step (2), a gRNA expression vector is constructed through a process comprising specifically: after gRNA synthesis, dissolving the primers in water to give a concentration of 10 μM; adding each 10 μL of the upstream and downstream primers; incubating at 95° C. for 10 min and then at room temperature for 30 min; ligating a scaffold, where the scaffold is a T vector with U6 promoter and gRNA tail and the restriction site is BsaI; and where after the scaffold was prepared, the scaffold is ligated by T4 ligase at 16° C. for 4 hrs.

6. The production method according to claim 3, wherein in Step (3), the cells are transiently transfected by CRISPR-Cas9 method, and the cleaving activity of the plasmid is verified by detecting the integrity of the target gene, through a process comprising:
(3.1) thawing PK15 porcine kidney cells in a total of 4 cells in a 24-well plate, and transiently transfecting according to the instructions of invitrogen lipofectamin 2000, where the cells in one well are transfected with an EGFP containing plasmid to observe the transfection efficiency; and the other three wells are co-transfected with the gRNA vector plasmid;
(3.2) determining the transfection with the target plasmid based on the intensity of fluorescence expression from PK15 porcine kidney cells 17 hrs after transfection with the EGFP containing plasmid; and
(3.3) 48 hrs after transfection, collecting the cells to extract DNA, and identifying the activity by digestion with T7 endonuclease,

| Reagent | Volume | |
|---|---|---|
| Sample | 17 | μL |
| T7 endonuclease I | 0.2 | μL |
| Buffer 2 | 2 | μL |
| H₂O | 0.8 | μL |
| Total volume | 20 | μL |

where the reaction procedure comprises digestion at 37° C. for 60 min.

7. The production method according to claim 3, wherein Step (4) comprises transfection of cells, wherein
all of the three gRNAs are verified to have the gene cleaving activity and applicable to the construction of gene knockout cell lines; when the SK-N-SH cells are cultured and grown to reach 80%-90% confluence, the plasmid is nucleofected by a nucleofector according to a ratio of gRNA1:gRNA2:gRNA3:cas9:pcDNA3.1=1:1:1:2:1, where the passage number of the cells is P3; the cell transfection is observed 24 hrs after transfection; when the confluence reaches 80%-90%, the cells are subcultivated, and screened with G418 on the following day, and a single clone is obtained 7 days later; the single clone is picked to culture in a 96-well plate, and expanded to culture in a 48-well plate when it is overgrown; and when the confluence is 80%-90%, half of the cells are digested into a lysate for identification by PCR, and the remaining cells are cultured in the original wells.

8. The production method according to claim 3, wherein Step (5) comprises
(5.1) identification of cell clones by digestion with T7EI endonuclease, wherein:
whether the cells are gene knockout cell clones is determined by digestion with T7 endonuclease; PCR amplification and digestion with T7 endonuclease are carried out; a 20 μl system is used for PCR amplification, 2 μl of the PCR product is dotted, and the remaining 18 μl is divided into two groups; 9 μl is mixed with water at a ratio of 1:1, and the other 9 μl is mixed with wild-type PCR product at a ratio of 1:1; after denaturation and annealing, the liquid is digested with T7 endonuclease, where if cleavable by direct digestion, the cells are heterozygous knockout cells, and if failed to be cleaved by direct digestion but cleavable by mixed digestion, the cells are homozygous knockout cells; one suspected positive clone RXR/KO-SK-N-SH-35 # is identified from this batch; and two suspected positive clones are identified from the other batch, which are RXR/KO-SK-N-SH-7 # and RXR/KO-SK-N-SH-15 # respectively;
(5.2) gene sequencing of suspected positive cell clones:
the PCR products of the cell lysates of suspected positive cell clones are ligated to a T vector and sequenced; and the results show that the genotypes of #7 and #35 are the same and clones #7 and #35 are both a homozygote in which the RXRα allele are deleted; #15 is a cell line in which the RXRα gene is dual knocked out, wherein the bases 61-122 (62 bp), and the bases 61-122 (62 bp), 61-70 (10 bp) and 114-141 (28 bp) on exon 4 of the RXRα gene are deleted respectively; and
(5.3) Identification of RXRα protein expression level in positive clone cells, wherein:
identification by Western Blot shows that the RXRα gene knockout cell line constructed in the present invention has decreased RXRα protein expression level in the cells after the RXRα gene is knocked out.

9. A RXRα gene knockout cell line with stable and low RXRα protein expression, which is RXR/KO-SK-N-SH-7 # or RXR/KO-SK-N-SH-35 #, and RXR/KO-SK-N-SH-15 # respectively, and obtained through the production method according to claim 2.
